(19) United States
(12) Patent Application Publication
(10) Pub. No.: US 2025/0207103 A1
(43) Pub. Date: Jun. 26, 2025

(54) CYTOCOMPATIBLE HYDROGELS AND METHODS OF MAKING AND USING THE SAME

(71) Applicant: The Trustees of Indiana University, Bloomington, IN (US)

(72) Inventors: Chien-Chi Lin, Indianapolis, IN (US); Fang-Yi Lin, Bloomington, IN (US)

(21) Appl. No.: 18/851,829

(22) PCT Filed: Apr. 25, 2023
(86) PCT No.: PCT/US2023/066188

§ 371 Date: Sep. 27, 2024

Related U.S. Application Data
(60) Provisional application No. 63/334,804, filed on Apr. 26, 2022.

(51) Int. Cl. C12N 5/09; C08F 20/70; C12N 5/00

(52) U.S. Cl. CPC C12N 5/0693; C08F 20/70; C12N 5/0012; C12N 2513/00; C12N 2533/30

(57) ABSTRACT

A composition comprising a hydrogel includes a polymer of formula (1), at least one repeating unit of which comprises BO₂H₂. Methods of making such hydrogels and methods of encapsulating cells using such hydrogels are also disclosed herein.

### CROSS-REFERENCE TO RELATED APPLICATIONS

This application claims priority to U.S. provisional patent application No. 63/334,804, filed on Apr. 26, 2022, which is incorporated by reference in its entirety herein.

### STATEMENT REGARDING FEDERALLY SPONSORED RESEARCH

This invention was made with government support under CA227737 awarded by National Institutes of Health. The Government has certain rights in the invention.

### FIELD

The present disclosure provides hydrogels and methods of making and using the same.

### BACKGROUND

Hydrogels have shown promise as matrices for three-dimensional cell culture. Cells tend to adhere, spread, migrate and proliferate better in hydrogels with pronounced stress relaxation.

### SUMMARY

A need exists, therefore, for mechanisms for tuning the stress relaxation and other viscoelastic behaviors of hydrogels.

The present disclosure provides a composition comprising a hydrogel. The hydrogel may include a polymer of formula (1):

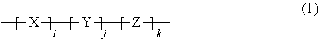

where X comprises one or more repeating units of formula (2):

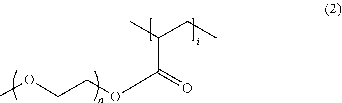

Y comprises one or more repeating units of formula (3):

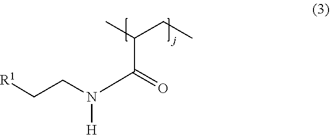

Z comprises one or more repeating units of formula (4):

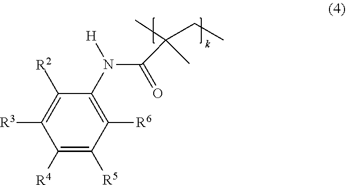

R¹ is H, OH, or a substituent of formula (5), where the substituent of formula (5) binds the repeating unit of formula (3) at a position labeled *:

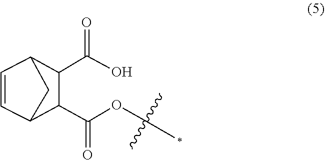

R²-R⁶ each independently comprise H, C, or a heteroatom; and n, i, j, and k, are each independently an integer from 2 to 4000; wherein at least one repeating unit of Z comprises BO₂H₂.

In embodiments at least one repeating unit of Y comprises the substituent of formula (5).

In embodiments, R²-R⁶ are each independently H, BO₂H₂, halogen, SO₃H, OH, NH₃, OR⁷, NO₂, CN, R⁷CN, C(O)R⁷, alkoxy, alkyl, alkyloxyalkyl, alkylaryl, aryl, alkenyl, arylalkyl, arylalkenyl, or alkylarylalkenyl. R⁷ is alkyl, monocyclic alkyl, bicyclic alkyl, polycyclic alkyl, monocyclic heterocycle, bicyclic heterocycle, polycyclic heterocycle, alkyloxyalkyl, alkylaminoalkyl, alkylaryl, aryl, heteroaryl, alkenyl, arylalkyl, arylalkenyl, or alkylarylalkenyl, and R⁷ is optionally substituted with other functional groups.

In embodiments, the other functional groups, when present, are selected from the group consisting of amino, carboxyl, hydroxy, alkoxy, ketone, aldehyde, halogen, and a combination of two or more thereof.

In embodiments, R² of at least one repeating of Z is BO₂H₂. In embodiments, R³ of at least one repeating of Z is BO₂H₂. In embodiments, R⁴ of at least one repeating of Z is BO₂H₂. In embodiments, R⁵ of at least one repeating of Z is BO₂H₂. In embodiments, R⁶ of at least one repeating of Z is BO₂H₂. In embodiments, R², R³, R⁴, and R⁶ of at least one repeating of Z are each H, and R⁵ of the at least one repeating of Z is BO₂H₂.

In embodiments, the composition further includes at least one pancreatic cancer cell.

In embodiments, the polymer of formula (1) comprises a number average molecular weight (M_{N}) obtained via gel permeation chromatography (GPC) from 15 kDa to 55 kDa.

In embodiments, a concentration of BO₂H₂ groups present on the polymer of formula (1) is from 0.25 mmol/g to 1 mmol/g.

In embodiments, the composition further comprises a thiol-containing molecule. In embodiments, the thiol-containing molecule is selected from the group consisting of 1,4-dithiothreitol, 4-arm thiolated polyethylene glycol (PEG), a peptide that includes more than one cysteine residues, a natural or thiolated protein, thiolated hyaluronic acid, a thiolated collagen, and a combination of two or more thereof. In embodiments, the thiol-containing molecule comprises a thiolated protein and the thiolated protein is thiolated gelatin.

Another aspect of the present disclosure provides a method for making a composition comprising a hydrogel includes forming a polymer of formula (1) via a radical process and conjugating a norbornene of formula (5) to at least one repeat unit of formula (3) to produce a norbornene-functionalized polymer of formula (1).

In embodiments, the method further includes adding at least one pancreatic cancer cell to the composition. In embodiments, the pancreatic cancer cell is from a mammal. In embodiments, the mammal is a human.

Another aspect of the present disclosure provides a method for encapsulating at least one cell includes forming the composition disclosed above, adding a thiol-containing molecule to the composition, adding the cell to the composition, and allowing the substituent of formula (5) to react with the thiol-containing molecule, thereby forming the hydrogel and encapsulating the cell.

In embodiments, the cell is a cancer cell. In embodiments, the cell is a pancreatic cancer cell. In embodiments, the pancreatic cancer cell is from a mammal. In embodiments, the mammal is a human.

In embodiments, allowing the substituent of formula (5) to react with the thiol-containing molecule comprises exposing the composition to electromagnetic energy.

This summary is not intended to identify key or essential features of the claimed subject matter, nor is it intended to be used as an aid in limiting the scope of the claimed subject matter. Further embodiments, forms, features, and aspects of the present disclosure shall become apparent from the description and figures provided herewith.

### BRIEF DESCRIPTION OF THE DRAWINGS

The teachings of some embodiments of the present disclosure will be better understood by reference to the description taken in conjunction with the accompanying drawings, wherein:

FIGS. 1A-1E show schemes of reversible addition-fragmentation chain transfer (RAFT) polymer synthesis and hydrogel crosslinking reactions according to embodiments. FIG. 1A shows a scheme of RAFT polymerization for synthesis of poly(HEAA) (PH), poly(OEGA-s-HEAA) (PEH), and poly(OEGA-s-HEAA-s-APBA) (PEHA). FIG. 1B shows a scheme of a synthesis of dopamine-decorated PHEAA (PHD). FIG. 1C shows a scheme of a synthesis ofnorbornene-decorated poly(OEGA-s-HEAA-s-APBA) (PEH_{NB}A). FIG. 1D shows a scheme of a light-mediated thiol-norbomene crosslinking. FIG. 1E shows a scheme of a reversible boronate ester bonding. “OEGA” refers to oligoethylene glycol acrylate; “HEAA” refers to N-hydroxyethyl acrylamide; and “APBA” refers to 3-aminophenylboronic acid.

FIG. 2 shows refractive index traces of polymers from gel permeation chromatograpghy (GPC) characterization according to embodiments.

FIGS. 3A-3C show ¹H NMR spectra of PEH_{NB} (FIG. 3A), PEH_{NB}A (FIG. 3B), and PHD (FIG. 3C) according to embodiments.

FIG. 4 shows rheological properties of thiol-norbornene RAFT polymer hydrogels according to embodiments. (A) In situ photo-rheometry of elastic (i, ii) and viscoelastic (iii) hydrogels. (B) Evolution of tan δ during in situ photo-gelation. (C) Effect of PHD content on G′ and tan δ. (D) Stress relaxation curves of hydrogels crosslinked by 3 wt % PEH_{NB}A, 3 wt % PEH_{NB}+2 wt % PHD, and 3 wt % PEH_{NB}A+2 wt % PHD. All gels contained 2 wt % PEG4SH. (E) Impact of PHD content on stress-relaxation of 3 wt % PEH_{NB}A hydrogels crosslinked by 1 wt % PEG4SH.

FIG. 5 shows results with cancer-associated fibroblast (CAF) cell-laden hydrogels according to embodiments. (A) Rheological properties of PEH_{NB} (or PEH_{NB}A)/GelSH/PEG4SH/PHD hydrogels. (B) Live/dead images on Day 14 stained by Ethidium homodimer III (red). (C) Cell morphological analysis on Day 14.

FIGS. 6A-6B shows representative images for morphology analyse according to embodiments. FIG. 6A shows an elastic hydrogel and FIG. 6B shows a viscoelastic hydrogel. Scale bar=200 μm. F-actin (red) and nuclei (blue) stained images.

FIGS. 7A-7D shows the effect of matrix viscoelasticity on pancreatic cancer cell (PCC)—cancer associated fibroblast (CAF) interactions according to embodiments. FIG. 7A shows PCCs-only in elastic or viscoelastic hydrogels. FIG. 7B shows PCCs and CAFs co-encapsulated in elastic or viscoelastic hydrogels. FIG. 7C shows the effect of myosin-inhibitor blebbistatin and the ROCK-inhibitor Y-27632 on spreading of CAFs in viscoelastic hydrogels FIG. 7D shows theeffect of blebbistatin and Y-27632 on PCCs/CAFs encapsulated in viscoelastic hydrogels (blebbistatin: 40 μM; Y-27632: 10 μM). Images taken on Day 15 post-encapsulation.

FIG. 8 shows PCC/CAF co-encapsulated hydrogels treated with 40 μM blebbistatin and 10 μM Y-27632 on D15 in separate channels according to embodiments. Blue: nuclei. Green: CAF. Red: PCC. White: F-actin. Scale bar: 100 μm.

FIGS. 9A-9B show gene expression in PCC-only and PCC/CAF co-encapsulated viscoelastic hydrogels according to embodiments. FIG. 9A shows upregulated and FIG. 9B shows downregulated genes in the co-encapsulated group.

FIG. 10 shows the effect of hydrogel viscoelasticity on PCC/CAF secrotomes according to embodiments. (A, B) growth factor. (C) matrix metalloproteinase (MMPs).

FIG. 11 shows human growth factor array results according to embodiments.

FIG. 12 shows human matrix metalloproteinase (MMP) array results according to embodiments.

### DETAILED DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

Unless defined otherwise, all technical and scientific terms have the same meaning as is commonly understood by one of ordinary skill in the art to which this disclosure belongs. All patents, applications, published applications and other publications are incorporated by reference in their entireties. If a definition set forth in this section is contrary to, or otherwise inconsistent with, a definition set forth in a patent, application, or other publication that is incorporated by reference, the definition set forth in this section prevails over the definition incorporated by reference.

As used in the description and the appended claims, the singular forms “a,” “an,” and “the” are intended to include the plural forms as well, unless the context clearly indicates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The terms “including,” “containing,” and “comprising” are used in their open, non-limiting sense. Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of ±20%, ±10%, ±5%, ±1%, ±0.5%, or even ±0.1% of the specified amount. To provide a more concise description, some of the quantitative expressions are not qualified with the term “about.” It is understood that, whether the term “about” is used explicitly or not, every quantity is meant to refer to the actual given value, and it is also meant to refer to the approximation to such given value that would reasonably be inferred based on the ordinary skill in the art, including equivalents and approximations due to the experimental and/or measurement conditions for such given value.

The terms “including,” “containing,” and “comprising” are used in their open, non-limiting sense. The transitional phrase “consisting essentially of” means that the scope of a claim is to be interpreted to encompass the specified materials or steps recited in the claim, “and those that do not materially affect the basic and novel characteristic(s)” of the claimed subject matter. See In re Herz, 537 F.2d 549, 551-52, 190 USPQ 461, 463 (CCPA 1976) (emphasis in the original); see also MPEP § 2111.03 (9^{th} edition, 10^{th} revision).

Certain features of the disclosure, which are, for clarity, described in the context of separate embodiments, may also be provided in combination in a single embodiment.

Conversely, various features of the disclosure, which are, for brevity, described in the context of a single embodiment, may also be provided separately or in any suitable subcombination. All combinations of the embodiments pertaining to the metabolic biomarkers represented by the variables are specifically embraced by the present disclosure just as if each and every combination was individually and explicitly disclosed. In addition, all subcombinations of the chemical groups listed in the embodiments describing such variables are also specifically embraced by the present disclosure just as if each and every such sub-combination of metabolic biomarkers was individually and explicitly disclosed.

References in the specification to “one embodiment,” “an embodiment,” “an illustrative embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. It should be further appreciated that although reference to a “preferred” component or feature may indicate the desirability of a particular component or feature with respect to an embodiment, the disclosure is not so limiting with respect to other embodiments, which may omit such a component or feature. Further, when a particular feature, structure, or characteristic is described in connection with an embodiment, it is submitted that it is within the knowledge of one skilled in the art to implement such feature, structure, or characteristic in connection with other embodiments whether or not explicitly described. Additionally, it should be appreciated that items included in a list in the form of “at least one of A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C). Further, with respect to the claims, the use of words and phrases such as “a,” “an,” “at least one,” and/or “at least one portion” should not be interpreted so as to be limiting to only one such element unless specifically stated to the contrary, and the use of phrases such as “at least a portion” and/or “a portion” should be interpreted as encompassing both embodiments including only a portion of such element and embodiments including the entirety of such element unless specifically stated to the contrary.

As used herein, the term “mammal” refers to, for example, humans, other primates (e.g., monkeys, chimpanzees, etc.), companion animals (e.g., dogs, cats, horses, etc.), farm animals (e.g., goats, sheep, pigs, cattle, etc.), laboratory animals (e.g., mice, rats, etc.), and wild and zoo animals (e.g., wolves, bears, deer, etc.).

The term “cancer,” as used herein, refers to any benign or malignant abnormal growth of cells. Examples include, without limitation, breast cancer, prostate cancer, lymphoma, skin cancer, pancreatic cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, acute lymphocytic leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma. In some embodiments, the cancer is selected from the group of tumor-forming cancers. In some embodiments, the cancer is pancreatic cancer.

Nuclear magnetic resonance (NMR) spectroscopy is a technique for determining the molecular structure and concentration of molecules. In the NMR analysis of compositions described herein, the compositions or one or more components thereof are maintained in solution and then placed in a large magnetic field. The magnetic field is typically generated by a cryogenically cooled superconducting magnet, but lower magnetic fields including those generated by room temperature electromagnets or rare earth magnets can also be used. The samples are exposed to a band of radiofrequency (RF) waves which are absorbed by the molecules and excite the nuclei of the molecules. The typical focus of these experiments is on the excitation of hydrogen nuclei (also referred to as protons) and nuclei of carbon, in particular the C-13 isotope. After the RF excitation is turned off, a receiver coil then detects the RF energy that is released by the nuclei as they return from the excited state. Following Fourier transformation of these signals, an NMR spectrum is generated containing spectral peaks that, in different combinations, represent the identity of the molecules in solution. The resulting NMR spectrum can then be compared with reference spectra to both identify and quantify the molecules in the solution.

Gel permeation chromatography (GPC), also sometimes interchangeably called gas permeation chromatography, is an analytical method to characterize molecular weights (MW) and dispersity index (Ð) of polymers. GPC is based on size-exclusion chromatography, where the polymers are loaded on the column and separated based on their hydrodynamic volume. Principly, porous beads are packed in the GPC column and the polymers are eluted by organic solvent through the column. Smaller polymers can enter the pores of the beads more easily than larger polymers, hence increasing their retention time in the column. As such, larger polymers are eluted out first while smaller polymers are retained in the column for a longer time. Based on the retention time and elution profiles, polymer molecular weights and dispersity can be calculated.

In an aspect of the disclosure, there is provided a composition comprising a hydrogel. The hydrogel includes a polymer of formula (1):

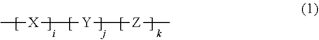

where X comprises one or more repeating units of formula (2):

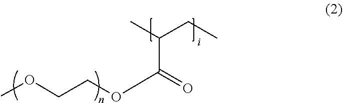

Y comprises one or more repeating units of formula (3):

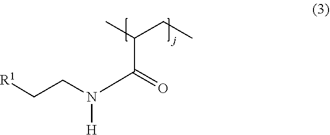

Z comprises one or more repeating units of formula (4):

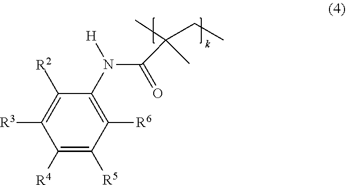

R¹ is H, OH, or a substituent of formula (5), where the substituent of formula (5) binds the repeating unit of formula (3) at a position labeled *:

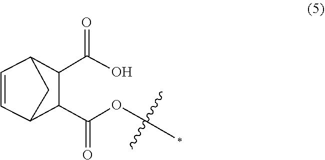

R²-R⁶ each independently comprise H, C, or a heteroatom; and n, i, j, and k, are each independently an integer from 1 to 5000; wherein at least one repeating unit of Z comprises BO₂H₂.

In embodiments, the polymer of formula (1) may be a random copolymer. In other embodiments, the polymer of formula (1) may be a block copolymer. As a random copolymer, X, Y, and Z, may be bonded to one another in any order. That is, in one portion of the polymer of formula (1), there may be one or more repeat units of X, followed by one or more repeat units of Y, followed by one or more repeat units of Z. In other portions of the polymer of formula (1), there may be one or more repeat units of Y, followed by one or more repeat units of X, followed by one or more repeat units of Z. In other portions of the polymer of formula (1), there may be one or more repeat units of Y, followed by one or more repeat units of X, followed by one or more repeat units of Y, followed by one or more repeat units of Z. In other portions of the polymer of formula (1), there may be one or more repeat units of X, followed by one or more repeat units of Y, followed by one or more repeat units of X, followed by one or more repeat units of Z. All other permutations of the binding order of X, Y, and Z are envisioned.

As noted above, R¹ is H, OH, or a substituent of formula (5), where the substituent of formula (5) binds the repeating unit of formula (3) at a position labeled *:

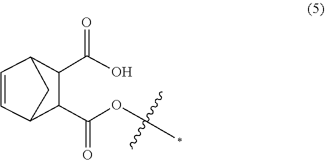

Without intending to be bound by any particular theory, it is believed that the norbornene-bearing substituent of formula (5) may provide a convenient pathway to thiol-norbomene click gelation. That is, a thiol-containing molecule may be included in the composition to facilitate gel formation. In some embodiments, the thiol-containing molecule comprises a multi-functional thiol. In some embodiments, the thiol-containing molecule includes one or more of 1,4-dithiothreitol, 4-arm thiolated PEG, a peptide that includes more than one cysteine residues, a natural or thiolated protein such as thiolated gelatin, thiolated hyaluronic acid, and thiolated collagens, and combinations thereof. In some embodiments, the dithiol molecule comprises dithiothreitol.

Additional components may be present in the composition. For instance, a diol may be added in certain embodiments. In embodiments, the diol may be a 1,2-diol. Exemplary 1,2-diols include, but are not limited to, 1,2-ethanediol, propane-1,2-diol, and dopamine. Furthermore, in some embodiments, the diol functionality may be installed on the polymer of formula (1). One convenient location for installing the diol functionality is at the terminal hydroxyl of Y of formula (1). That is, when R¹ is OH, the OH may be reacted with an appropriate diol, such as dopamine, either before or after the polymerization forming the polymer of formula (1).

In embodiments, R²-R⁶ each independently comprise H, C, or a heteroatom. However, at least one of R²-R⁶ is BO₂H₂. In embodiments, R²-R⁶ may each independently be H, BO₂H₂, halogen, SO₃H, OH, NH₃, OR⁷, NO₂, CN, R⁷CN, C(O)R⁷, alkoxy, alkyl, alkyloxyalkyl, alkylaryl, aryl, alkenyl, arylalkyl, arylalkenyl, or alkylarylalkenyl. In embodiments, R⁷ is alkyl, monocyclic alkyl, bicyclic alkyl, polycyclic alkyl, monocyclic heterocycle, bicyclic heterocycle, polycyclic heterocycle, alkyloxyalkyl, alkylaminoalkyl, alkylaryl, aryl, heteroaryl, alkenyl, arylalkyl, arylalkenyl, or alkylarylalkenyl, and R⁷ is optionally substituted with other functional groups. In embodiments, the other functional groups may include amino, carboxyl, hydroxy, alkoxy, ketone, aldehyde, halogen, and a combination of two or more thereof.

In embodiments, R² is BO₂H₂. In embodiments, R³ is BO₂H₂. In embodiments, R⁴ is BO₂H₂. In embodiments, R⁵ is BO₂H₂. In embodiments, R⁶ is BO₂H₂. In embodiments, R², R³, R⁴, and R⁶ are each H, and R⁵ is BO₂H₂. Without intending to be bound by any particular theory, it is believed that the BO₂H₂ groups may facilitate dynamic covalent bonding via formation of boronate ester bonds with diols.

In embodiments, the concentration of the BO₂H₂ groups present on the polymer of formula (1) is from 0.25 mmol/g to 1 mmol/g. That is, the concentration of the BO₂H₂ groups present on the polymer of formula (1) is from 0.25 mmol/g to 0.95 mmol/g, from 0.25 mmol/g to 0.90 mmol/g, from 0.25 mmol/g to 0.85 mmol/g, from 0.25 mmol/g to 0.8 mmol/g, from 0.25 mmol/g to 0.75 mmol/g, from 0.25 mmol/g to 0.7 mmol/g, from 0.25 mmol/g to 0.65 mmol/g, from 0.25 mmol/g to 0.6 mmol/g, from 0.25 mmol/g to 0.55 mmol/g, from 0.25 mmol/g to 0.5 mmol/g, from 0.25 mmol/g to 0.45 mmol/g, from 0.25 mmol/g to 0.4 mmol/g, from 0.25 mmol/g to 0.35 mmol/g, from 0.25 mmol/g to 0.3 mmol/g, from 0.3 mmol/g to 0.95 mmol/g, from 0.35 mmol/g to 0.95 mmol/g, from 0.4 mmol/g to 0.95 mmol/g, from 0.45 mmol/g to 0.95 mmol/g, from 0.5 mmol/g to 0.95 mmol/g, from 0.55 mmol/g to 0.95 mmol/g, from 0.6 mmol/g to 0.95 mmol/g, from 0.65 mmol/g to 0.95 mmol/g, from 0.7 mmol/g to 0.95 mmol/g, from 0.75 mmol/g to 0.95 mmol/g, from 0.8 mmol/g to 0.95 mmol/g, from 0.85 mmol/g to 0.95 mmol/g, or even from 0.9 mmol/g to 0.95 mmol/g. Without intending to be bound by any particular theory, it is believed that a lower concentration of BO₂H₂ groups may lead to a low degree of stress-relaxation. Further, it is believed that a higher concentration of BO₂H₂ groups may reduce the solubility of the polymer.

In embodiments, n, i, j, and k, are each independently an integer from 1 to 5000. For example, n, i, j, and k, may each independently be an integer from 1 to 4500, 1 to 4000, 2 to 4000, 1 to 3500, from 1 to 3000, from 1 to 2500, from 1 to 2000, from 1 to 1500, from 1 to 1000, from 1 to 500, from 1 to 450, from 1 to 400, from 1 to 350, from 1 to 300, from 1 to 250, from 1 to 200, from 1 to 150, from 1 to 100, from 1 to 50, from 50 to 5000, from 100 to 5000, from 150 to 5000, from 200 to 5000, from 250 to 5000, from 300 to 5000, from 350 to 5000, from 400 to 5000, from 450 to 5000, from 500 to 5000, from 1000 to 5000, from 1500 to 5000, from 2000 to 5000, from 2500 to 5000, from 3000 to 5000, from 3500 to 5000, from 4000 to 5000, or even from 4500 to 5000.

In embodiments, the polymer of formula (1) may have a number average molecular weight (M_{N}), obtained via GPC, from 15 kDa to 55 kDa. That is, the M_{N} may be from 20 kDa to 55 kDa, from 25 kDa to 55 kDa, from 30 kDa to 55 kDa, from 35 kDa to 55 kDa, from 40 kDa to 55 kDa, from 45 kDa to 55 kDa, from 50 kDa to 55 kDa, from 15 kDa to 50 kDa, from 15 kDa to 45 kDa, from 15 kDa to 40 kDa, from 15 kDa to 35 kDa, from 15 kDa to 30 kDa, from 15 kDa to 25 kDa, or even from 15 kDa to 20 kDa. Without intending to be bound by any particular theory, it is believed that hydrogel crosslinking may be inefficient if the M_{N} is too low. Further, it is believed that too high of a M_{N} may limit the usability of hydrogels in vivo.

In another aspect of the disclosure, there is provided a method for making a composition comprising a hydrogel. The pmethod includes forming a polymer of formula (1) via a radical process and conjugating a norbornene of formula (5) to at least one repeating unit of Y to produce a norbornene-functionalized polymer of formula (1).

In embodiments, the radical process is reversible addition-fragmentation chain transfer (“RAFT”). In embodiments, an initiator is used to initiate the RAFT polymerization. Initiators are known. In embodiments, the initiator may be an azo series catalyst including, but not limited to, 4′-Azobis-4-cyanovaleric acid (ACVA), 2′-Azobis-isobutyronitrile (AIBN), Azobis-2,4-dimethylvaleronitrile (ADVN), and 2′-Azobis-2-methylbutyronitrile (AMBN). In addition to the initiator, the RAFT polymerization may also use a RAFT agent. Commonly used RAFT agents include thiocarbonylthio compounds such as dithioesters, dithiocarbamates, trithiocarbonates, and xanthates. A literature review regrding RAFT polymerization is available in Perrier, “50^{th} Anniversary Perspective: RAFT Polymerization—A User Guide,” Macromolecules, vol. 50, pp. 7433-47 (2017), the entire content of which is incorporated herein by reference.

In embodiments, the method for making a composition comprising a hydrogel includes adding at least one cancer cell to the composition. In embodiments, the cancer cell is a pancreatic cancer cell. In embodiments, the pancreatic cancer cell is from a mammal. In embodiments, the pancreatic cancer cell is from a human.

In embodiments, the composition comprising the hydrogel may have an elastic modulus (G′) from 2 kPa to 10 kPa. That is, G′ may be from 2 kPa to 9 kPa, from 2 kPa to 8 kPa, from 2 kPa to 7 kPa, from 2 kPa to 6 kPa, from 2 kPa to 5 kPa, from 2 kPa to 4 kPa, from 2 kPa to 3 kPa, from 3 kPa to 10 kPa, from 4 kPa to 10 kPa, from 5 kPa to 10 kPa, from 6 kPa to 10 kPa, from 7 kPa to 10 kPa, from 8 kPa to 10 kPa, from 9 kPa to 10 kPa. Without intending to be bound by any particular theory, it is believed that an undesirably weak hydrogel may be obtained when G′ is too low. Further, it is believed that may be too stiff if G′ is too high. Hydrogels that are either too weak or too stiff are not physiologically relevant.

In embodiments, the composition comprising the hydrogel may have a tan δ from 0.01 to 0.1. That is, the tan δ may be from 0.01 to 0.09, from 0.01 to 0.08, from 0.01 to 0.07, from 0.01 to 0.06, from 0.01 to 0.05, from 0.01 to 0.04, from 0.01 to 0.03, from 0.01 to 0.02, from 0.02 to 0.1, from 0.03 to 0.1, from 0.04 to 0.1, from 0.05 to 0.1, from 0.06 to 0.1, from 0.07 to 0.1, from 0.08 to 0.1, or even from 0.09 to 0.1. Without intending to be bound by any particular theory, tan δ is a measurement of hydrogel viscoelasticity. Similar to G′, there is an optimal range for soft tissues. Materials with too low or too high tan δ do not mimic tissues in the body, and are therefore not physiologically relevant.

In embodiments, the composition comprising the hydrogel may have a stress-relaxation halftime (t_{1/2}) from 10 seconds to 100 seconds. That is, the t_{1/2} may be from 10 seconds to 90 seconds, from 10 seconds to 85 seconds, from 10 seconds to 80 seconds, from 10 seconds to 75 seconds, from 10 seconds to 70 seconds, from 10 seconds to 65 seconds, from 10 seconds to 60 seconds, from 10 seconds to 55 seconds, from 10 seconds to 50 seconds, from 10 seconds to 45 seconds, from 10 seconds to 40 seconds, from 10 seconds to 35 seconds, from 10 seconds to 30 seconds, from 10 seconds to 25 seconds, from 10 seconds to 20 seconds, from 10 seconds to 15 seconds, from 20 seconds to 100 seconds, from 25 seconds to 100 seconds, from 30 seconds to 100 seconds, from 35 seconds to 100 seconds, from 40 seconds to 100 seconds, from 45 seconds to 100 seconds, from 50 seconds to 100 seconds, from 55 seconds to 100 seconds, from 60 seconds to 100 seconds, from 65 seconds to 100 seconds, from 70 seconds to 100 seconds, from 75 seconds to 100 seconds, from 80 seconds to 100 seconds, from 85 seconds to 100 seconds, from 90 seconds to 100 seconds, or even from 95 seconds to 100 seconds. Without intending to be bound by any particular theory, t_{1/2} is a measurement of hydrogel viscoelasticity. Similar to G′ and tan δ, there is an optimal range for soft tissues. Materials with too low or too high t_{1/2} do not mimic tissues in the body, and are therefore not physiologically relevant.

In another aspect of the disclosure, there is provided a method for encapsulating at least one cell. The method includes forming any embodiment of the composition disclosed above and then adding a thiol-containing molecule to the composition. Further, the cell is added to the composition. Notably, the thiol-containing compound and cell may be added to the composition concurrently or sequentially. That is, the thiol-containing compound and cell may be added at the same time, the thiol-containing compound may be added first, or the cell may be added first. Once the thiol-containing compound and cell are both added to the composition, the norbomene substituent of formula (5) can then be allowed to react with the thiol-containing molecule, thereby forming the hydrogel, which encapsulates the cell.

In some embodiments, the polymer composition and the thiol-containing molecule are mixed together in various proportions ranging from 100:1 to 1:100 before initiating a photo-gelation reaction to yield a hydrogel. That is, the polymer composition and the diol-containing molecule may be mixed together in a proportion including 100:1, 95:1, 90:1, 85:1, 80:1, 75:1, 70:1, 65:1 60:1, 55:1, 50:1, 45:1, 40:1, 35:1, 30:1, 25:1, 20:1, 15:1, 10:1, 5:1, 1:1, 1:5, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, up to 1:100, before initiating a photo-gelation reaction to yield a hydrogel. The proportions of each component may be determined based upon the stoichiometric proportions of reactive groups necessary to optimize the reactivity of the reactants, and other practical considerations that are well within the purview of a person of ordinary skill in the art.

In some embodiments, the thiol-containing molecule comprises a multi-functional thiol. In some embodiments, the thiol-containing molecule molecule includes one or more of 1,4-dithiothreitol, 4-arm thiolated PEG, a peptide that includes more than one cysteine residues, a natural or thiolated protein such as thiolated gelatin, thiolated hyaluronic acid, and thiolated collagens, and combinations thereof. In some embodiments, the dithiol molecule comprises dithiothreitol.

In embodiments, the thiol-containing molecule is allowed to react with the norbornene substituent of formula (5) via a photo-initiated reaction. That is, in embodiments, gel formation may take place by exposing a composition including both the norbornene substituent of formula (5) and the thiol-containing molecule to electromagnetic radiation. In embodiments, the electromagnetic radiation comprises visible light, i.e., light at a wavelength from about 380 nm to about 760 nm. In embodiments, the electromagnetic radiation comprises ultraviolet light, i.e., light at wavelength from about 10 nm to about 400 nm. In embodiments, the electromagnetic radiation may include both visible light and ultraviolet light.

In addition to the aspects and embodiments described and provided elsewhere in the present disclosure, the following non-limiting list of embodiments are also contemplated.

1. A composition comprising a hydrogel, the hydrogel comprising a polymer of formula (1):

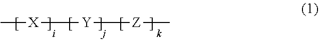

where
   X comprises one or more repeating units of formula (2):

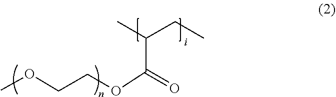

Y comprises one or more repeating units of formula (3):

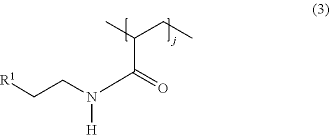

Z comprises one or more repeating units of formula (4):

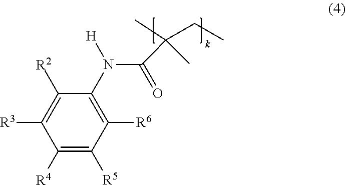

R¹ is H, OH, or a substituent of formula (5), where the substituent of formula (5) binds the repeating unit of formula (3) at a position labeled *.

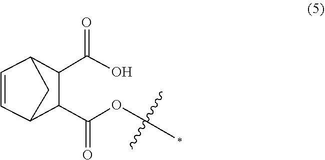

R²-R⁶ each independently comprise H, C, or a heteroatom; and
n, i, j, and k, are each independently an integer from 1 to 4000;
wherein
   at least one repeating unit of Z comprises BO₂H₂.
      2. The composition of clause 1, wherein at least one repeating unit of Y comprises the substituent of formula (5).
      3. The composition of clause 1 or clause 2, wherein R²-R⁶ are each independently H, BO₂H₂, halogen, SO₃H, OH, NH₃, OR⁷, NO₂, CN, R⁷CN, C(O)R⁷, alkoxy, alkyl, alkyloxyalkyl, alkylaryl, aryl, alkenyl, arylalkyl, arylalkenyl, or alkylarylalkenyl;
where R⁷ is alkyl, monocyclic alkyl, bicyclic alkyl, polycyclic alkyl, monocyclic heterocycle, bicyclic heterocycle, polycyclic heterocycle, alkyloxyalkyl, alkylaminoalkyl, alkylaryl, aryl, heteroaryl, alkenyl, arylalkyl, arylalkenyl, or alkylarylalkenyl, and R⁷ is optionally substituted with other functional groups.
   4. The composition of clause 3, wherein the other functional groups, when present, are selected from the group consisting of amino, carboxyl, hydroxy, alkoxy, ketone, aldehyde, halogen, and a combination of two or more thereof.
   5. The composition of any one preceding clause, wherein R² of at least one repeating of Z is BO₂H₂.
   6. The composition of any one preceding clause, wherein R³ of at least one repeating of Z is BO₂H₂.
   7. The composition of any one preceding clause, wherein R⁴ of at least one repeating of Z is BO₂H₂.
   8. The composition of any one preceding clause, wherein R⁵ of at least one repeating of Z is BO₂H₂.
   9. The composition of any one preceding clause, wherein R⁶ of at least one repeating of Z is BO₂H₂.
   10. The composition of clause 8, wherein R², R³, R⁴, and R⁶ of at least one repeating of Z are each H, and R⁵ of the at least one repeating of Z is BO₂H₂.
   11. The composition of any one preceding clause further comprising at least one pancreatic cancer cell.
   12. The composition of any one preceding clause, wherein the polymer of formula (1) comprises a number average molecular weight (M_{N}) obtained via gel permeation chromatography (GPC) from 15 kDa to 55 kDa.
   13. The composition of any one preceding clause, wherein a concentration of BO₂H₂ groups present on the polymer of formula (1) is from 0.25 mmol/g to 1 mmol/g.
   14. The composition of any one preceding clause, further comprising a thiol-containing molecule.
   15. The composition of clause 14, wherein the thiol-containing molecule is selected from the group consisting of 1,4-dithiothreitol, 4-arm thiolated PEG, a peptide that includes more than one cysteine residues, a natural or thiolated protein, thiolated hyaluronic acid, a thiolated collagen, and a combination of two or more thereof.
   16. The composition of clause 15, wherein the thiol-containing molecule comprises a thiolated protein and the thiolated protein is thiolated gelatin.
   17. A method for making a composition comprising a hydrogel, the method comprising: forming a polymer of formula (1) via a radical process:

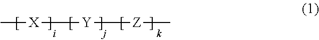

where
   X comprises one or more repeating units of formula (2):

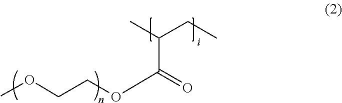

Y comprises one or more repeating units of formula (3):

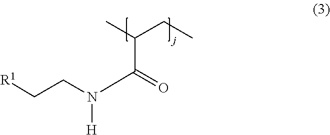

Z comprises one or more repeating units of formula (4):

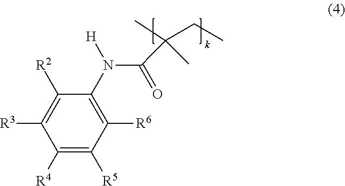

R¹ is H or OH;
R²-R⁶ each independently comprise H, C, or a heteroatom;
n, i, j, and k, are each independently an integer from 1 to 4000;
wherein
   at least one repeating unit of Z comprises BO₂H₂; and
conjugating a norbomene of formula (5) to at least one repeating unit of Y to produce a norbomene-functionalized polymer of formula (1), where the substituent of formula (5) is substituted for R¹ via a position labeled *:

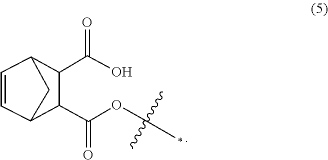

18. The method of clause 17, further comprising:
adding at least one pancreatic cancer cell to the composition.
   19. The method of clause 18, wherein the pancreatic cancer cell is from a mammal.
   20. The method of clause 19, wherein the mammal is a human.
   21. A method for encapsulating at least one cell, the method comprising:
forming the composition of any one of clauses 2-16;
adding a thiol-containing molecule to the composition;
adding the cell to the composition; and
allowing the substituent of formula (5) to react with the thiol-containing molecule, thereby forming the hydrogel and encapsulating the cell.
   22. The method of clause 21, wherein the cell is a cancer cell.
   23. The method of clause 21 or clause 22, wherein the cell is a pancreatic cancer cell.
   24. The method of clause 23, wherein the pancreatic cancer cell is from a mammal.
   25. The method of clause 24, wherein the mammal is a human.
   26. The method of any one of clauses 21-25, wherein allowing the substituent of formula (5) to react with the thiol-containing molecule comprises exposing the composition to electromagnetic energy.

### EXAMPLES

Examples related to the present disclosure are described below. In most cases, alternative techniques can be used. The examples are intended to be illustrative and are not limiting or restrictive of the scope of the invention as set forth in the claims.

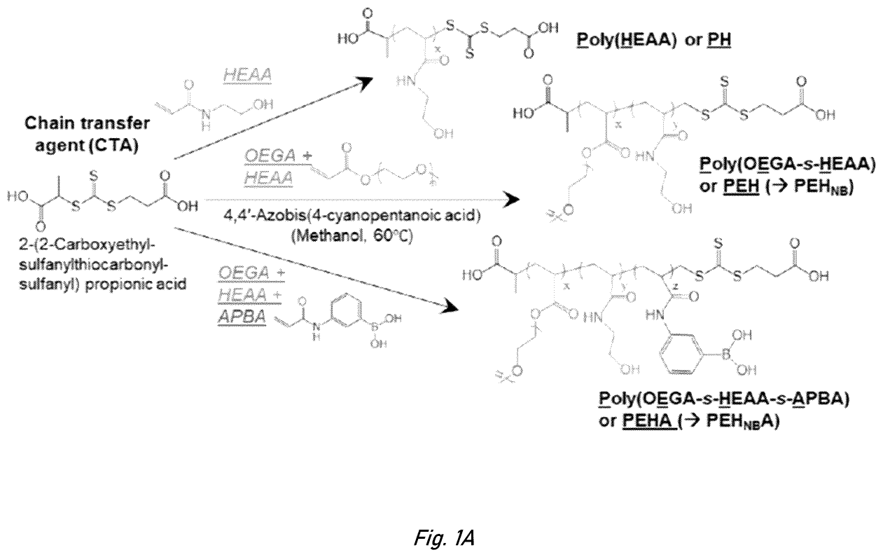

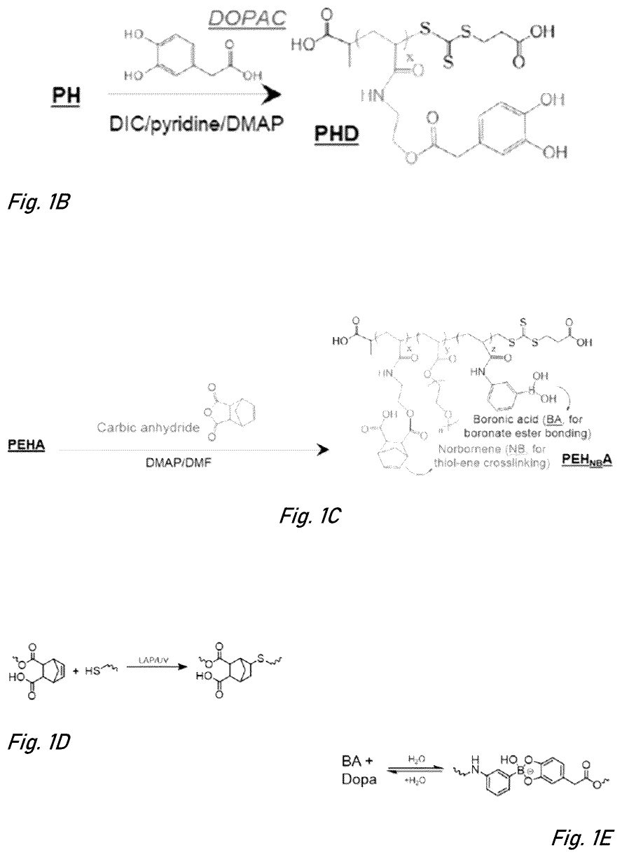

To study the design of new compositions comprising a polymer of formula (1), a new macromer poly(oligo(ethylene glycol) acrylate-s-hydroxyethyl acrylate-s-acrylamidophenyl-boronic acid (“poly(OEGA-s-HEAA-s-APBA” or “PEHA”) was synthesized. As shown in FIGS. 1A-1E, PEHA contains pendant boronic acid groups for forming boronate-diol bonding with diol-containing molecules. Additionally, norbomene (NB) was conjugated to PEHA via hydroxyl pendant groups on poly(N-hydroxyethylacrylamide) (also called polyacrylate-s-hydroxyethyl” or “HEAA”), rendering a PEH_{NB}A macromer susceptible to both thiol-norbornene crosslinking and boronate-diol bonding. Using PEH_{NB}A, semi-interpenetrating (“semi-IPN”) hydrogels were formed with tunable shear moduli (G′) and degrees of stress-relaxation. Viscoelastic RAFT polymer hydrogels were used to study the effect of matrix mechanics on epithelial-mesenchymal transition (EMT) phenotype in pancreatic cancer associated fibroblasts (CAF) and pancreatic cancer cells (PCC).

### Materials and Methods

2-(2-Carboxyethylsulfanylthiocarbonylsulfanyl)propionic acid (CPA, 95%, Sigma-Aldrich), methanol (ACS reagent, 99.8%, Sigma-Aldrich), N,N′-diisopropylcarbodiimide (DIC, Chem-Impex), 3,4-dihydroxyphenylacetic acid (DOPAC, Ambeed), 4-dimethylaminopyridine (DMAP, Aldrich), dimethylformamide (DMF, anhydrous, Alfa Aesar), lithium phenyl-2,4,6-trimethylbenzoylphosphinate (LAP, ≥95%, Sigma-Aldrich), 4-arm thiolated PEG (PEG4SH, 10 kDa, Laysan Bio), and pyridine (Fisher) were used as received. 4,4′-Azobis(4-cyanovaleric acid) (ACVA, 98%, Sigma-Aldrich) was recrystallized from methanol. Oligo(ethylene glycol) methyl ether acrylate (OEGA, M_{N}˜480 g/mol, Sigma-Aldrich) and N-hydroxyethyl acrylamide (HEAA, 97%, Sigma-Aldrich) were passed through the inhibitor remover column prior to conducting the polymerization. 3-acrylamidophenylboronic acid (AAPBA) was obtained by reacting 3-aminophenylboronic acid with acryloyl chloride using known procedures. Thiolated gelatin (GelSH) was synthesized using gelatin, ethylene diamine, and Traut's reagent. The thiol concentration of GelSH was 0.22 mmol/g as determined by Ellman's assay.

Data were presented as mean±standard error of the mean (SEM). One-way ANOVA was used to determine the statistical significance between groups when p<0.05.

### Example 1: Polymer Synthesis and Characterization

PHEAA, PEH, and PEHA polymers were synthesized via RAFT polymerization. For an exemplary PEHA synthesis, CPA (50 mg, 194 μmol), HEAA (1.3 g, 11.67 mmol, 60 equiv.), AAPBA (1.5 g, 7.78 mmol, 40 equiv.), OEGA (7 g, 14.58 mmol, 75 equiv.), ACVA (10.9 mg, 39 μmol, 0.2 equiv.), and methanol (19.7 g) were charged in a reaction vial with a stir bar. The reaction was purged under nitrogen for 15 minutes followed by reacting at 60° C. for 8 hours.

The product was dialyzed in regenerated cellulose (molecular weight cut-off (MWCO)=3.5 k) bag against pure water for 3 days and subsequently lyophilized with yield ≥90%. The feed ratio of [CTA]:[HEAA]:[AAPBA]:[OEGA]:[ACVA] was set at 1:220:0:0:0.2 for PHEAA, 1:60:0:91:0.2 for PEH, and 1:60:40:75:0.2 for PEHA, respectively.

The reaction conversion and final composition was examined by ¹H NMR (Bruker, 500 MHz) in deuterated DMSO-d₆.

Prior to molecular weight characterization by gel permeation chromatography (GPC), the boronic acid of PEHA was protected by pinacol using conventional proceedures to eliminate potential interaction between polymers and GPC columns. Briefly, pinacol and PEHA were combined in a reaction vessel and heated to 60° C. The reaction continued under agitation for 1 hour. The product was dialyzed using MWCO=3.5 kDa regenerated cellulose membrane in methanol for 1 day. The protected product was recovered after solvent evaporation.

The number-average molecular weight of polymers and the corresponding dispersity were characterized by Agilent 1100 high-performance liquid chromatography (HPLC) system from Cambridge Polymer Group. The system was run at 40° C. in dimethylformamide (DMF) with 0.10% lithium bromide at a flow rate of 1 mL/min. The system was equipped with a UV/Vis detector and a refractive index detector, 3 Agilent PLgel Mixed-C columns and calibrated by poly(methyl methacrylate) standards. Samples were prepared at 2 mg/mL and passed through a 0.45 μm polytetrafluoroethylene (PTFE) filter before testing.

The concentration of boronic acid in the final product of PEHA was determined by alizarin red (ARS) using a conventional protocol. Briefly, the content of boronic acid in PEHA was determined using AAPBA monomer as the standard. AAPBA standard solutions (starting from 500 μM) and PEHA sample were prepared in phosphate buffer solution (PBS). Alizarin red S sodium salt (ARS) was prepared in PBS at 0.3 mM. In a 96-well microplate, 125 μL ARS solution was combined with equal volume of standard or sample (n=2). The solution was incubated at room temperature for 30 minutes and read between 400-800 nm at 1 nm interval. The concentration-dependent λₘₐₓ was shifted from 515 nm to 456 nm under different level of ARS and boronic acid complexation.

PEH and PEHA polymers were conjugated with norbomene functional groups to afford PEH_{NB} and PEH_{NB}A as thiol-norbomene photocrosslinking macromers. Using a conventional protocol, carbic anhydride was conjugated onto polymers through the hydroxyl groups of HEAA. Briefly, PEHA (1 g, [OH]=1.2 mmol), carbic anhydride (0.97 g, 5.9 mmol, 5 equiv.), DMAP (73 mg, 594 μmol, 0.5 equiv.) and 10 mL DMF were combined in a reaction vial equipped with a stir bar. The reaction was conducted for 8 hours at 100° C. The product was dialyzed in pure water for 3 days and characterized by ¹H NMR spectra in deuterated DMSO-d₆ or deuterium oxide.

The norbomene concentration was quantified by Ellman's assay. Briefly, the calibration curve of norbomene concentration was constructed by exposing 200 μL solution containing 4 mM cysteine, 2 mM LAP, and 0-2 mM carbic anhydride under UV light (λ=365 nm, 5 mW/cm²) for 2 minutes. The residual cysteine concentration was quantified by regular Ellman's assay. To determine norbomene concentration in PEH_{NB} and PEH_{NB}A, 200 μL solution containing 4 mM cysteine, 2 mM LAP, and 0-10 mg/mL polymer was exposed under UV light for the same procedure.

PHEAA polymers were conjugated with dopamine functional groups to enable the boronate ester chemistry in the hydrogel. Dopamine was conjugated onto PHEAA through the acid group of DOPAC and the hydrogel group of PHEAA. DOPAC (1.81 g, 10.8 mmol, 2.5 equiv.) was activated in 18 mL DMF by DIC (1.69 mL, 10.8 mmol, 2.5 equiv.) under nitrogen environment for 1 hours in dark. Meanwhile, PHEAA (0.5 g, [OH]=4.3 mmol), DMAP (263 mg, 2.2 mmol, 0.5 equiv.), and pyridine (1.74 mL, 21.5 mmol, 5 equiv.) were dissolved in 5 mL DMF. The activated DOPAC was then transferred into PHEAA solution. The reaction was continued overnight under nitrogen environment in dark. The product was dialyzed in methanol for 1 day followed by 2 days in pure water. The conjugation efficiency was analyzed by ¹H NMR spectra in deuterated DMSO-d₆.

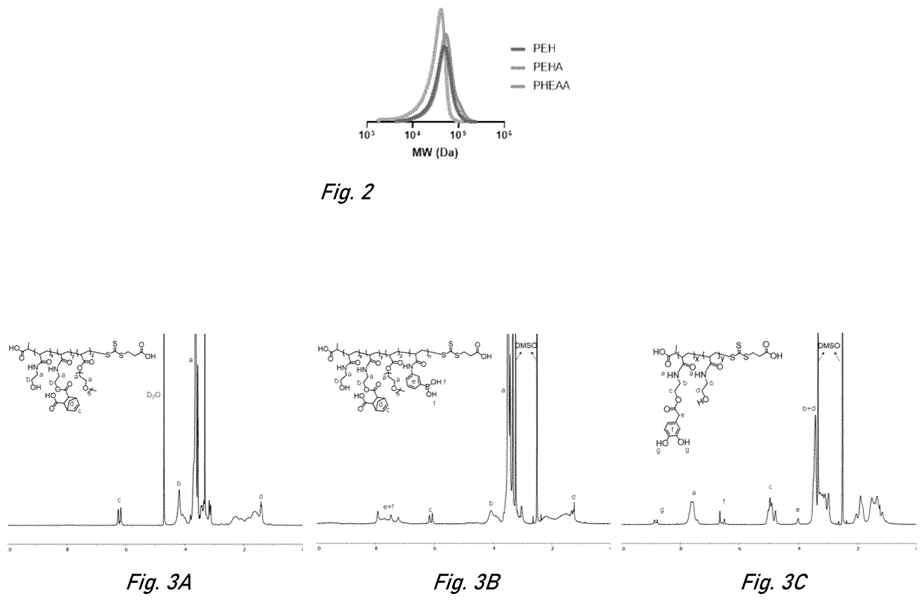

Discussion: Using only three monomers, OEGA, HEAA, and APBA, three sets of RAFT polymers were synthesized: PHEAA, poly(OEGA-s-HEAA) (PEH), and poly(OEGA-s-HEAA-s-APBA) (PEHA), as shown in FIG. 1. Post-synthesis, PHEAA was modified by DOPAC to afford dopamine-functionalized PHEAA (PHD), as shown in FIGS. 1A-1E, which provided 1,2-diols for forming boronate-ester-diol bonds with network immobilized BA moieties. PEH was used as a BA-free control polymer. Finally, BA-containing PEHA was further conjugated with carbic anhydride to afford PEH_{NB}A, a dually functionalized linear polymer amenable to orthogonal thiol-norbornene polymerization and boronate-ester-diol bonding, as shown in FIG. 1C. The 3 polymers were analyzed by GPC, as shown in FIG. 2 and summarized in Table 1, infra, and ¹H NMR spectra, as shown in FIGS. 3A-3C. Polymers revealed number-average molecular weight (M_{N}) between 23.5 and 39.6 kDa with dispersity of 1.32-1.45. Due to the overlap of HEAA and OEGA ester peaks at δ3.0-4.3, NMR spectra only gave qualitative results. The concentration of boronic acid in PEHA was therefore quantified by its strong complexation with ARS, which induced color change from red to yellow when complexing, using conventional methods. It was determined that PEHA contains 0.54 mmol/g boronic acid. In terms of norbornene, the norbomene concentration of PEH_{NB} and PEH_{NB}A was quantified by the modified Ellman's assay. The assay determines the residual cysteine amount from the reaction between excess cysteine and norbornene. The norbornene concentration of PEH_{NB} and PEH_{NB}A is 0.40 and 0.31 mmol/g, respectively, as summarized in Table 1, infra. For PHD, without the presence of OEGA, dopamine concentration can be determined by NMR spectra, as shown in FIG. 3C. The conjugation efficiency was 20% and afforded dopamine concentration of 1.91 mmol/g. The concentration of the various functional groups is summarized in Table 1.

TABLE 1

| | RAFT | | | Dopamine | Norbornene | Boronic Acid |
|---|---|---|---|---|---|---|
| | Conversion | M_{N} | | Concentration | Concentration | Concentration |
| Polymer | (%) | (KDa) | Dispersity | (mmol/g) | (mmol/g) | (mmol/g) |
| PHD | 90 | 23.5 | 1.45 | 1.91 | N/A | N/A |
| PEH_{NB} | 81 | 35.8 | 1.32 | N/A | 0.4 | N/A |
| PEH_{NB}A | 94 | 39.6 | 1.38 | N/A | 0.31 | 0.54 |

### Example 2: Hydrogel Fabrication

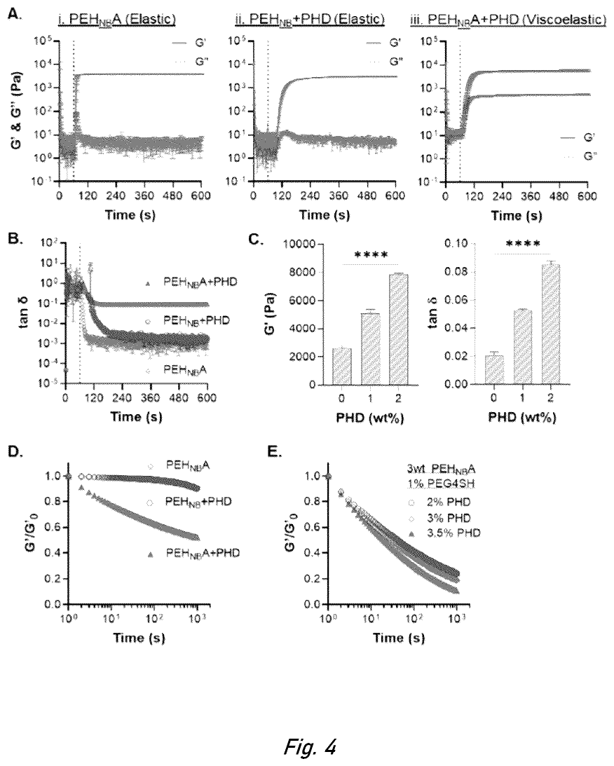

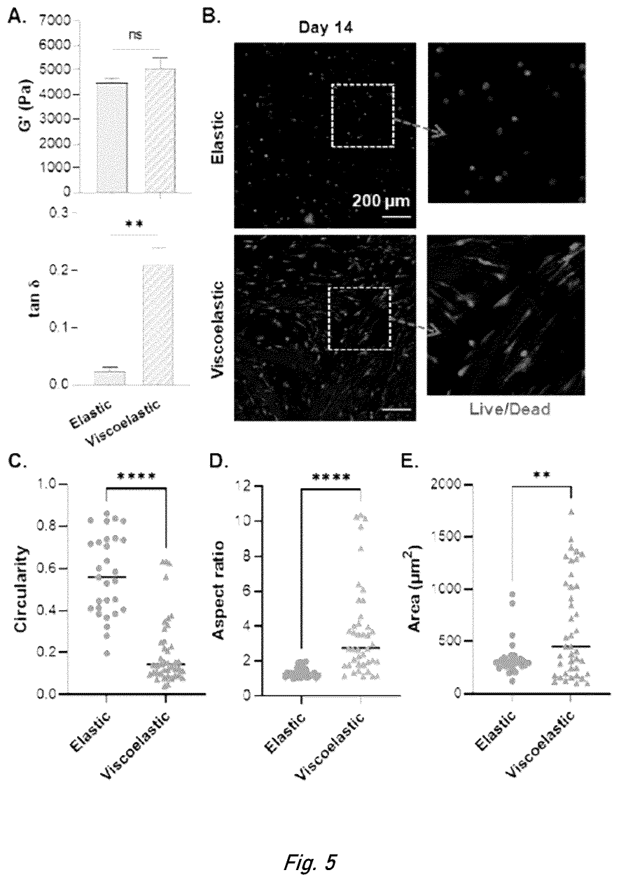

Detailed hydrogel formulations are summarized in Table 2, and these formulations were used to obtain the data summarized in FIG. 4 and FIG. 5.

TABLE 2

| | | PEH_{NB} | PEH_{NB}A | PEG4SH | GelSH | PHD |
|---|---|---|---|---|---|---|
| FIG. | Sample | wt % | wt % | wt % | wt % | wt % |
| 4 | PEH_{NB}A/PEG4SH | 0 | 3 | 2 | 0 | 0 |
| 4 | PEH_{NB}/PEG4SH/PHD | 3 | 0 | 2 | 0 | 2 |
| 4 | PEH_{NB}A/PEG4SH/PHD | 0 | 3 | 2 | 0 | 2 |
| 4 (panel E) | PEH_{NB}A/PEG4SH/PHD | 0 | 3 | 1 | 0 | Varies |
| 5 | Elastic | 3 | 0 | 0.5 | 3 | 2 |
| 5 | Viscoelastic | 0 | 3 | 0 | 3 | 2 |

For rheological testing, a predetermined amount of PEH_{NB} or PEH_{NB}A, PEG4SH, PHD, and LAP precursor solutions were mixed in PBS. Fifty μL precursor solution was deposited between 8 mm parallel plates with a gap size of 800 μm on Anton Paar MCR102 for in situ gelation acquisition. Moduli data (n=3) were acquired at 1 Hz with UV light (λ=365 nm, 5 mW/cm²) beginning at t=60 s for a total 10-minute acquisition period. The amplitude sweep and stress relaxation measurement were conducted on pre-fabricated hydrogels. That is, 60 μL precursor solution was deposited between glass slides with 1 mm spacer and crosslinked under UV light (λ=365 nm, 5 mW/cm²) for 2 minutes to afford hydrogel. After overnight incubation at 37° C. in PBS, the shear moduli of hydrogels (n=3) were determined by amplitude sweep between the strain of 0.1% and 5% at 1 Hz with 0.25 N normal force at 25° C. between 8 mm parallel plates. The stress relaxation curve was measured using an initial strain of 10% for a total 15-minute acquisition.

Discussion: The norbornene groups on PEH_{NB}A were used for orthogonal hydrogel crosslinking with multi-functional thiol linker (e.g., 4-arm PEGSH). Rapid crosslinking of PEH_{NB}A-based hydrogels (see FIGS. 1A-1E) was demonstrated by in situ photorheometry with PEG4SH as the crosslinker and LAP as the photoinitiator. Specifically, gel points were 8, 39, and 13 seconds for hydrogels crosslinked by PEH_{NB}A, PEH_{NB}+PHD, and PEH_{NB}A+PHD hydrogels, respectively, as shown in panel A of FIG. 4. From panel A of FIG. 4, it was apparent that the addition of PHD in PEH_{NB}A hydrogels contributed to the increase of viscous moduli and the corresponding tan δ, as shown in panel B of FIG. 4. These increases were the result of the fast equilibrium between dopamine and boronic acid functional groups (see FIG. 1E), which dissipated energy when subjected to the constant strain during stress relaxation. Furthermore, changing PHD content in the precursor formulation (i.e., from 0 wt % to 2 wt %) resulted in hydrogels with higher elastic moduli (G′, increased from ˜2.3 kPa to ˜8 kPa) and tan δ (increased from ˜0.02 to ˜0.085), an indication that the inclusion of PHD contributed to not only the viscous but elastic properties of the hydrogels, as shown in panel C of FIG. 4. Finally, stress-relaxation tests were performed to realize the viscoelastic nature of semi-IPN PEH_{NB}A+PHD hydrogels, as shown in panel E of FIG. 4. Only hydrogels containing PEG_{NB}A and PHD showed apparent stress-relaxation, but the relaxation halftime (t_{1/2}) was ˜1,000 seconds owing to the use of high PEG4SH content (2 wt %) that led to high hydrogel G′ (˜8 kPa, see panel C of FIG. 4). To further demonstrate that the stress-relaxation of PEH_{NB}A-based hydrogels could be tuned in a physiologically relevant range, the PEG4SH concentration was lowered to 1 wt % and PHD content was adjusted. As expected, hydrogel stress-relaxation of these hydrogels was much faster than that of gels crosslinked with 2 wt % PEG4SH. Specifically, the t_{1/2} was 42, 29, and 20 seconds for 2 wt %, 3 wt %, and 3.5 wt % PHD, respectively, confirming the highly tunable viscoelastic nature of the PEH_{NB}A semi-IPN hydrogels.

### Example 3: Cancer Associated Fibroblast and Pancreatic Cancer Cell Encapsulation

To facilitate imaging, pancreatic cancer cells COLO-357 were stably transduced with mKate2, a red fluorescent protein (RFP), using INCUCYTE® Cytolight Lentivirus reagent, followed by puromycin selection. green fluorescent protein (GFP)-labelled human pancreatic cancer associated fibroblasts (1303-GFP-49-hT, or CAF) and RFP-labelled COLO-357 (PCC) were maintained in high glucose Dulbecco's modified eagle medium (DMEM, Gibco) containing 10% Fetal bovine serum (FBS, Corning) and 1% antibiotic-antimycotic (Gibco) at 37° C. with 5% CO₂. Cells were trypsinized by 0.1% trypsin-ethylenediaminetetraacetic acid (trypsin-EDTA) (Gibco) for cell encapsulation. The cell density of CAF and PCC was kept at 1.6 million/mL (M/mL) and 400 thousand/mL (k/mL), respectively, in both single-encapsulated and co-encapsulated precursor solutions. The hydrogel precursor solution was similar to the aforementioned formulations, except that 3 wt % of GelSH was included to facilitate cell proliferation. Twenty-five μL precursor solution was cured under UV light (λ=365 nm, 5 mW/cm²) in an opened 1 mL syringe for 2 minutes. The cell-laden hydrogels were then cultured in regular cell culture media. For hydrogels treated with myosin inhibitor blebbistatin (40 μM, Peprotech) and ROCK inhibitor Y-27632 (10 μM, AdipoGen), the treated culture media was replaced daily to ensure the efficacy of inhibitors.

Discussion: Owing to the orthogonal thiol-norbomene crosslinking, it was possible to adjust hydrogel formulations to achieve independent control of G′ and tan δ. In one example, thiolated gelatin (GelSH) was used as the main multifunctional thiol crosslinker to afford both thiol groups for hydrogel crosslinking and to permit cell adhesion and protease-mediated degradation. The viscoelastic hydrogel was fabricated from PEH_{NB}A and GelSH in the presence of 2 wt % PHD. The elastic moduli was ˜4.5 kPa, while tan δ was calculated to be 0.2, as shown in panel A of FIG. 5. For the elastic hydrogel, additional 0.5 wt % PEGSH was added to the precursor solution to match the elastic moduli to its viscoelastic analogue. Since the extra PEGSH contributed exclusively to elastic hydrogel crosslinking, the tan δ of the elastic hydrogels remained low at ˜0.02. Additional stress-relaxation tests showed that the t_{1/2} for this hydrogel was ˜56 seconds (data not shown).

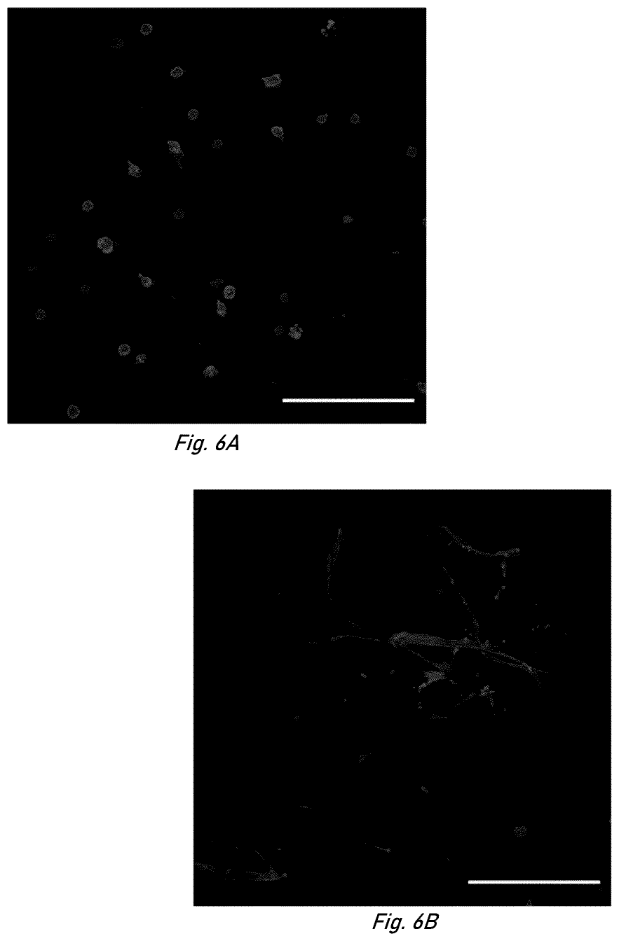

The addition of GelSH permitted cell-mediated matrix degradation, which is important for spreading and migration of many mesenchymal cells, including cancer-associated fibroblasts (CAFs). Regardless of the use of PEH_{NB} or PEH_{NB}A (i.e., elastic or viscoelastic hydrogel), the encapsulated CAFs remained highly viable with a very limited number of dead cells one day after in situ encapsulation (data not shown). After 14 days of in vitro culture, CAFs remained alive but with very limited spreading in elastic hydrogels, as shown in panel B of FIG. 5. In contrast, cells exhibited spindle shape morphology in viscoelastic hydrogels, as shown in panel B of FIG. 5. Quantitative analyses of the cell morphology demonstrated that cells in viscoelastic hydrogels exhibited significantly lower circularity, higher aspect ratio, as well as higher cell area than that in elastic hydrogels, as shown in panel C of FIG. 5 and in FIGS. 6A-6B.

### Example 4: Cellular and Molecular Analyses of Cell Fate

The cell viability of CAF in hydrogels was evaluated by staining with ethidium homodimer 3. The morphology of CAF in hydrogels was analyzed using F-actin (rhodamine phalloidin 100 nM, Cytoskeleton) and 4′,6-diamidino-2-phenylindole (DAPI) (1:1000, Anaspec) stained images. In the co-encapsulated hydrogels, cells were stained using F-actin (Actin stain 670 phalloidin 200 nM, Cytoskeleton) and DAPI (1:1000). The confocal images (Olympus Fluoview FV100 laser scanning microscope) were z-stacked from 5 μm slices over 100 μm in total.

The potential metastasis of PCC in co-encapsulated viscoelastic hydrogels was analyzed by gene expression. On day 12, hydrogels were degraded by chymotrypsin (2 mg/mL, Worthington). The RNA of the recovered cells was extracted using the RNA micro-scale kit (Thermo Fisher Scientific). 200 ng RNA of each condition was used to prepare cDNA using the cDNA synthesis kit (Thermo Fisher Scientific). Reverse transcription polymerase chain reaction (RT-PCR) was performed using the human tumor metastasis TAQMAN® array (Thermo Fisher Scientific). The kit contains 92 genes associated with tumor metastasis and 4 candidate endogenous control genes. 18 S was selected as endogenous control gene in this study. Additionally, the conditioned media between day 9 and day 12 was collected to investigate the secretomes under different conditions. The collected media was analyzed by human growth factor antibody array C1 (RayBiotech) and human matrix metalloproteinase (MMP) antibody array C1 (RayBiotech). The membranes were imaged by LAS3000 (Fuji Film).

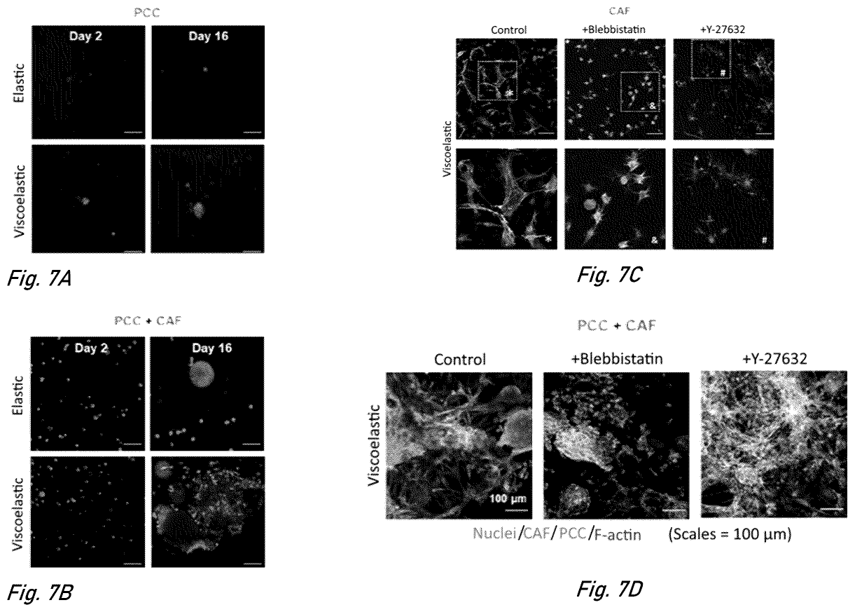

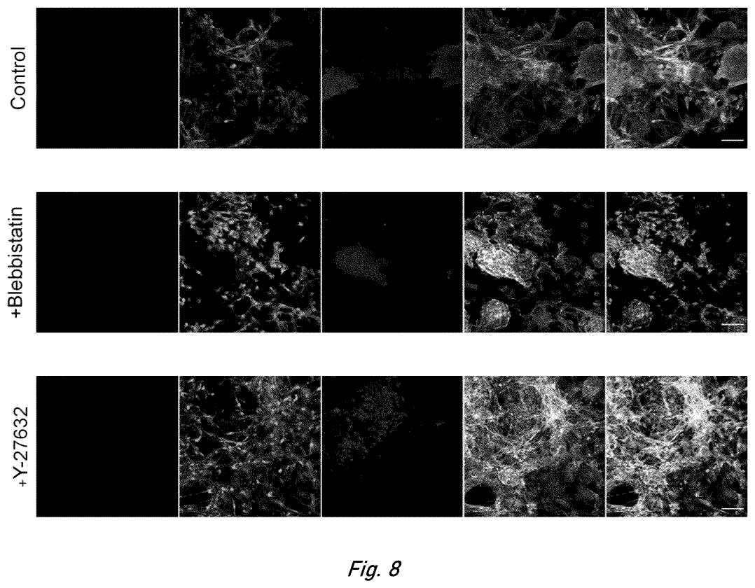

Discussion: COLO-357 cells, a pancreatic cancer cell (PCC) line derived from a metastasis of a pancreatic ductal adenocarcinoma (PDAC) tissue, was used to assess the effect of matrix viscoelasticity on PCC growth and potential epithelial-mesenchymal transition (EMT) in the absence or presence or CAFs. The encapsulated PCC cells showed minimal proliferation in the rather stiff (G′˜6 kPa) and elastic hydrogels, as shown in FIG. 7A, while cells in the stiff and viscoelastic hydrogels formed very small clusters after 16 days of culture, as shown FIG. 7A. When CAFs were co-encapsulated with PCC cells in the stiff elastic hydrogels, large PCC cell spheroids were visible in some area of the gels, but the two cell types remained separated, as shown in FIG. 7B. Strikingly, when CAF and PCC cells were co-encapsulated in the stiff and viscoelastic hydrogels, significant cancer cell outgrowth and irregular shapes of cell clusters were noted, as shown in FIG. 7B. Viscoelastic hydrogels were further used to probe the spreading of CAFs and PCC cells. Of note, F-actin staining was performed to visualize the spreading of cells in the hydrogels. As expected, the encapsulated CAFs showed extensive spreading in the viscoelastic hydrogels, as shown in FIG. 7C. Blebbistatin, a myosin II inhibitor, and ROCK inhibitor Y-27632 both disrupted F-actin polymerization, as shown in FIG. 7C, whereas the use of Y-27632 led to more obvious dendritic extensions of cells. Interestingly, neither the addition of blebbistatin nor Y-27632 altered invasive phenotype of PCC cells in the co-culture group, as shown in FIG. 7D and in FIG. 8.

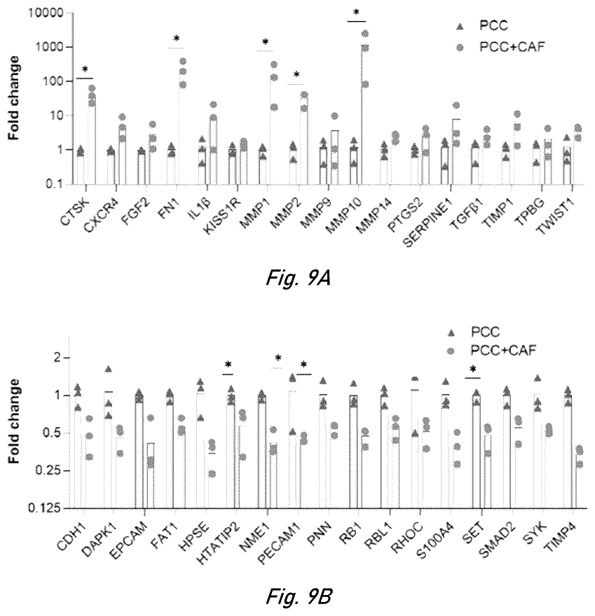

TAQMAN® Array Human Tumor Metastasis was used to screen for the potential molecular pathways leading to extensive spreading of PCCs in viscoelastic hydrogels in the presence of CAFs. In this study, 18 S was selected as the endogenous control gene since its expression was relatively stable (Ct values between 18.18 and 19.28) across all experimental conditions. The relative expression of all other genes was first normalized to endogenous control gene, then to the PCC only group. The expression of 92 genes between the control (PCC only) and experimental group (PCC+CAF) was organized into three groups: (1) increased expression with 2-fold or higher in the experimental group, as shown in FIG. 9A; (2) decreased expression with 0.5-fold or lower in the experimental group, as shown in FIG. 9B; and (3) similar expression between the two groups (data not shown). Co-culture in viscoelastic hydrogels led to significant increases in the expression of Cathepsin K (CTS), extracellular protein fibronectin (FN1), as well as various MMPs, including MMP1, MMP2, and MMP10, as shown in FIG. 9A. Co-culture in viscoelastic hydrogels also led to significant decreases in the expression of tumor metastasis-suppressor genes NME1, PNN, and RB1, as well as protease inhibitor TIMP4, as shown in FIG. 9B.

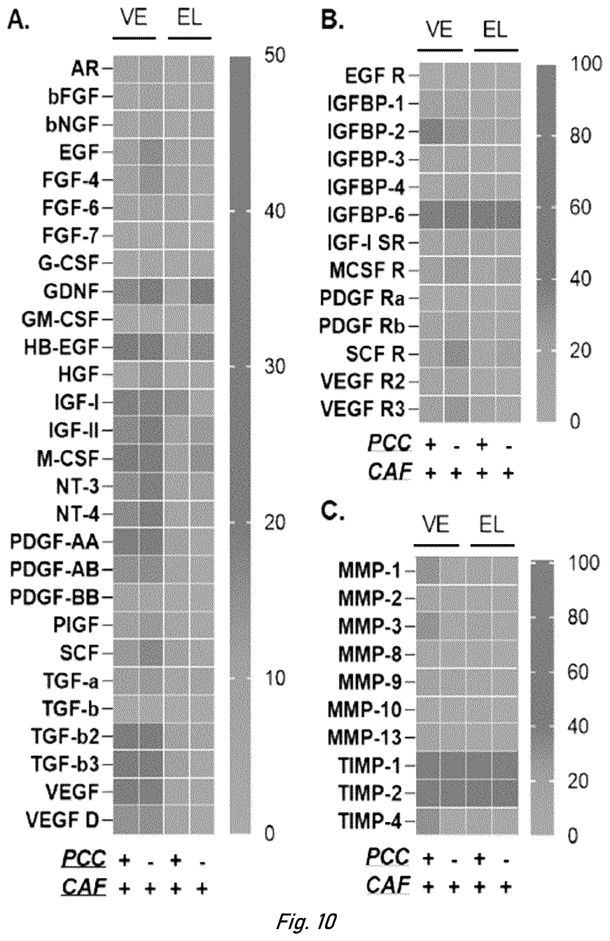

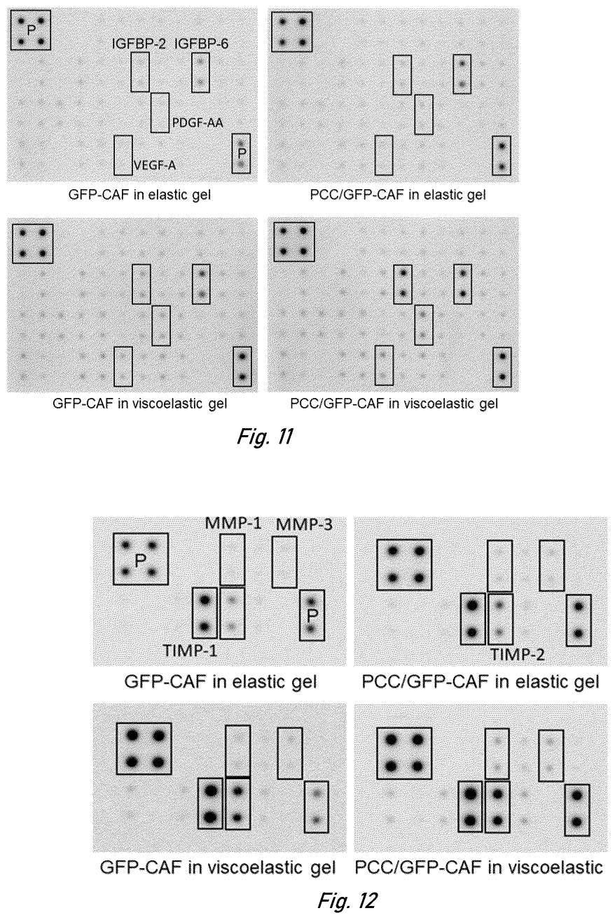

Multiplexed protein detection antibody arrays were used to assess conditioned media produced from CAF single culture or CAF/PCC co-culture in elastic and viscoelastic hydrogels. A total of 92 cell-secreted proteins (42 cytokines, 40 inflammatory factors, and 10 MMPs/TIMPs) were detected, and the protein spots were further analyzed by IMAGEJ to obtain semi-quantitative information about the level of secretion, as shown in FIG. 10, FIG. 11, and FIG. 12. In general, viscoelastic hydrogels promoted secretion of cytokines, inflammatory factors, and MMPs/TIPMs from both CAF single culture and CAF/PCC co-culture. Focusing on the CAF/PCC co-culture groups, numerous proteins were secreted 2-fold or higher in viscoelastic hydrogels than in elastic hydrogels, most notably HB-EGF (23.3 vs. 11.0), IGFBP-2 (100 vs. 12.9), M-CSF (21.4 vs. 10), PDGF-AA (45.6 vs. 9.5), TGF-β2 (24.3 vs. 9.5), TGF-β3 (26.7 vs. 10), MCSF-R (21.4 vs. 8.6), SCF-R (21.4 vs. 9.5), VEGF (30.1 vs. 5.2), and VEGF-R³ (21.8 vs. 10). Among these proteins, IGFBP-2, PDGF-AA, and VEGF were secreted ˜5-fold or higher.

### Example 5: Conclusions

Matrix stiffness and viscoelasticity are parameters in designing cell-laden biomaterials, as biological tissues exhibit different levels of stress-relaxation. While studies have shown that the elasticity of pancreatic stromal tissues would become progressively stiffer during pancreatic disease progression, the changes of matrix viscoelasticity (or stress-relaxation) was less studied. Previous work characterized the mechanical properties of resected human pancreatic tissues using an indentation-based tool. That work showed that, while the steady state moduli of PDAC tumor tissues (5.46±3.18 kPa) were significantly higher than that of normal tissues (1.06±0.25 kPa), the differences in viscosity (q) and characteristic relaxation halftime (t_{1/2}) were not significantly different for normal and tumor tissues (i.e., normal tissue: η=252±134 kPa·s; t_{1/2}=92.7±46.4 s; tumor tissue: η=349±222 kPa·s; t_{1/2}=66.1±20.8 s). On the other hand, the viscosity and stress-relaxation halftime of PanIN tissues were significantly lower than that of both normal and tumor tissues (η=63.2±26.7 kPa·s; t_{1/2}=27.6±14.0 s). The complex mechanical properties of pancreatic tissues in different stages of disease progression warrant the development of a biomaterial system capable of decoupling matrix stiffness from stress-relaxation.

Disclosed herein is a gelatin based semi-IPN hydrogel system to independently control matrix stiffness and viscoelasticity. To increase the tunability of hydrogel viscoelasticity, RAFT polymerization was used to synthesize polymers with defined chemical structures and functional group concentrations. This was achieved using three simple monomers: HEA, OEGA, and APBA. Homopolymerization of HEAA afforded PHEAA, while co-polymerization of OEGA/HEAA or OEGA/HEAA/APPA produced PEH or PEHA, respectively. Post-synthesis conjugation of DOPAC to PHEAAH yielded PHD, a dopamine-containing polymer that provided 1,2-diols for complexing with BA groups. On the other hand, post-synthesis modification of PEH or PEHA with carbic anhydride gave rise to PEH_{NB} or PEH_{NB}A, respectively. PEH_{NB}A was a dually functionalized polymer amenable to thiol-norbornene click gelation and boronate ester diol bonding, while PEH_{NB} was used as a control, BA-free polymer. PEH_{NB}A (or PEH_{NB}) was crosslinked into hydrogels by PEG4SH (or GelSH) via light-initiated thiol-norbornene click reaction. BA groups of PEH_{NB}A chains readily formed boronate-ester diols with linear PHD co-encapsulated in the hydrogels. The advantage of this hydrogel system is on the adaptability of the polymer formulations. For example, shear moduli of the hydrogels can be controlled by adjusting the content of PEG4SH (i.e., tuning the thiol-norbornene ratio) without affecting the compositions of other macromers. Furthermore, the degree of hydrogel stress-relaxation may be pre-determined by synthesizing PEH_{NB}A with different APBA monomer content or engineered via using PHD with different DOPAC substitution. In this study, viscoelastic hydrogels were produced with high elastic moduli (G′˜6 kPa) and fast relaxation halftime of ˜20 to ˜50 seconds, both of which were on par with the values reported in human PDAC tumor tissue elsewhere.

Using the highly tunable RAFT polymer hydrogels, it was demonstrated that high matrix viscoelasticity promoted spreading of pancreatic CAFs in 3D, whereas cells in the elastic hydrogels remained rounded. Furthermore, matrix viscoelasticity also impacted cancer cell growth and stromal-cancer cell interactions. This was apparently clear in PCCs grown in stiff hydrogels exhibiting different stress-relaxation. Specifically, in stiff and elastic hydrogels, PCCs alone did not grow well but their proliferation was significantly promoted when CAFs were co-encapsulated. Without intending to be bound by any particular theory, limited CAF-PCC interaction was observed in elastic hydrogels, suggesting the growth promoting effect was likely a result of paracrine signaling. However, when both cell types were co-encapsulated in viscoelastic hydrogels, close interactions between PCCs and CAFs were notable, with PCCs exhibiting invasive phenotype. The phenotypic changes of PCCs/CAFs were corroborated by significant increases in mRNA expression of protease Cathepsin K (CTSK), cell-adhesive protein fibronectin 1 (FN1) and MMPs (MMP-1, 2, 10). Both CTSK and MMPs are implicated in matrix degradation, while FN1 was increasingly deposited in tumor tissues. Furthermore, many genes implicated in tumor and metastasis suppression also downregulated in PCC/CAF co-cultured in viscoelastic gels (e.g., NME1, PNN, RB1).

Since elastic hydrogels did not induce PCC invasive phenotype even in the presence of CAFs, it was hypothesized that high matrix viscoelasticity likely promoted secretion of cytokines and other proteins that then induced PCC invasion into the matrix, although limitation to any particular theory is not intended. Indeed, the secretion of many growth- and EMT-promoting cytokines were promoted from PCC/CAF co-culture in viscoelastic hydrogels (e.g., HB-EGF, PDGF-AA, TGF-β2, TGF-β2, and IGFBP-2). Of note, overexpression of TGF-β2 in pancreatic cancer has been linked to immunosuppression, metastasis, angiogenesis, and proliferation. Furthermore, growing evidence indicates that IGFBP-2 in cancer cells is a “hub” for an oncogenic network, which connects multiple cancer signaling pathways. For example, IGFBP-2 is believed to activate the NF-κB pathway to drive EMT and invasive phenotype in PDAC. IGFBP-2 also promotes tumor progression by inducing alternative polarization of macrophages in PDAC through the STAT3 pathway. Co-culture also promoted the secretion of VEGF, and VEGF-R3, as well as proteases responsible for matrix degradation (MMP-1, 2, 3, 9, 13), a prerequisite for cell spreading and invasion. Of note, both TIMP-1 and TIMP-2 were overexpressed in PDAC tissues. While the major function of TIMPs is to inhibit MMPs, their expression was typically coordinated with the level of proteases. It was also hypothesized that the production of inhibitors may be a cellular reaction to the increased presence of proteases. Upregulation of TIMP-1 mRNA expression was correlated with aggressive tumors and the results presented herein comport with this effect. Moreover, high levels of TIMP-1 or TIMP-2 protein have been associated with poor prognosis. On the other hand, TIMP-2 may be growth-stimulating or growth-inhibiting, depending on their concentrations and the cell types.

Disclosed herein was a linear acrylate/acrylamide copolymer platform that contains norbornene and boronic acid moieties for hydrogel fabrication. The fabrication of synthetic semi-IPN hydrogels was through thiol-norbornene photocrosslinking chemistry and boronate ester equilibrium between phenylboronic acid and dopamine. This synthetic hydrogel system allowed independent control of the stiffness and viscoelasticity (or stress relaxation). The hydrogel stiffness and viscoelasticity were carefully regulated to mimic the pancreatic tumor microenvironment at elastic modulus ˜6 kPa, with or without stress relaxation characteristic for pancreatic cancer cells COLO-357 and pancreatic cancer-associated fibroblasts cell encapsulation. At this condition, CAFs only spread in viscoelastic hydrogels while they kept the circular shape in the elastic hydrogels. The encapsulated PCC cells showed minimal proliferation. However, CAF facilitated the proliferation of PCC in both elastic and viscoelastic hydrogels during co-encapsulation. While the two cell types remained intact in elastic condition, irregular cell clusters with outgrowing cancer cells were found in the viscoelastic condition. This cell behavior implied that high matrix viscoelasticity promotes tumor metastasis. The interactions between cells in viscoelastic hydrogels was also investigated by examining the cells' gene expression and cell secretion. The results indicated that PCCs exhibited invasive phenotype by significantly increasing cell-adhesive protein (FN1) along with aggressively degrading hydrogel matrix (MMP-1, 2, 10), while downregulating tumor and metastasis suppression (NME1, PNN, RB1). The investigation of secretomes further identified several cytokines, inflammatory factors, and MMPs that may promote PCC invasion into the matrix. The high overexpression of IGFBP-2, PDGF-AA, VEGF, and various MMPs (MMP-1, 2, 3, 9, 13) marked cell spreading and invasion of co-encapsulated cells in viscoelastic hydrogel. This study demonstrated that extensive cancer cell invasion assisted by cancer-associated fibroblasts can be regulated by hydrogel viscoelasticity. This system has the potential to serve as the in vitro model for screening and testing anti-cancer drugs.

While embodiments have been disclosed hereinabove, the present invention is not limited to the disclosed embodiments. Instead, this application is intended to cover any variations, uses, or adaptations of the invention using its general principles. Further, this application is intended to cover such departures from the present disclosure as come within known or customary practice in the art to which this invention pertains and which fall within the limits of the appended claims.

1. A composition comprising a hydrogel, the hydrogel comprising a polymer of formula (1): 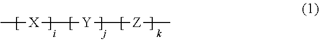
where
X comprises one or more repeating units of formula (2): 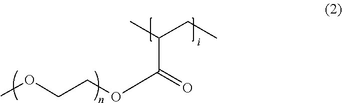
Y comprises one or more repeating units of formula (3): 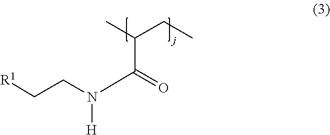
Z comprises one or more repeating units of formula (4): 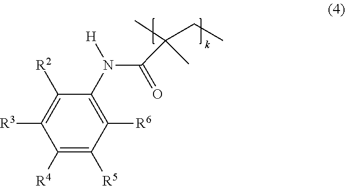
R^{t} is H, OH, or a substituent of formula (5), where the substituent of formula (5) binds the repeating unit of formula (3) at a position labeled *: 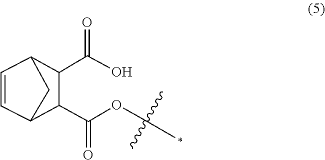
R²-R⁶ each independently comprise H, C, or a heteroatom; and
n, i, j, and k, are each independently an integer from 1 to 4000;
wherein
at least one repeating unit of Z comprises BO₂H₂.

2. The composition of claim 1, wherein at least one repeating unit of Y comprises the substituent of formula (5).

3. The composition of claim 1, wherein R²-R⁶ are each independently H, BO₂H₂, halogen, SO₃H, OH, NH₃, OR⁷, NO₂, CN, R⁷CN, C(O)R⁷, alkoxy, alkyl, alkyloxyalkyl, alkylaryl, aryl, alkenyl, arylalkyl, arylalkenyl, or alkylarylalkenyl;
where R⁷ is alkyl, monocyclic alkyl, bicyclic alkyl, polycyclic alkyl, monocyclic heterocycle, bicyclic heterocycle, polycyclic heterocycle, alkyloxyalkyl, alkylaminoalkyl, alkylaryl, aryl, heteroaryl, alkenyl, arylalkyl, arylalkenyl, or alkylarylalkenyl, and R⁷ is optionally substituted with other functional groups.

4. The composition of claim 3, wherein the other functional groups, when present, are selected from the group consisting of amino, carboxyl, hydroxy, alkoxy, ketone, aldehyde, halogen, and a combination of two or more thereof.

5. The composition of claim 1, wherein at least one of R², R³, R⁴, R⁵, or R⁶ of at least one repeating of Z is BO₂H₂.

6. (canceled)

7. (canceled)

8. (canceled)

9. (canceled)

10. The composition of claim 8, wherein R², R³, R⁴, and R⁶ of at least one repeating of Z are each H, and R⁵ of the at least one repeating of Z is BO₂H₂.

11. The composition of claim 1, further comprising at least one pancreatic cancer cell.

12. The composition of claim 1, wherein the polymer of formula (1) comprises a number average molecular weight (M_{N}) obtained via gel permeation chromatography (GPC) from 15 kDa to 55 kDa.

13. The composition of claim 1, wherein a concentration of BO₂H₂ groups present on the polymer of formula (1) is from 0.25 mmol/g to 1 mmol/g.

14. The composition of claim 1, further comprising a thiol-containing molecule.

15. The composition of claim 14, wherein the thiol-containing molecule is selected from the group consisting of 1,4-dithiothreitol, 4-arm thiolated PEG, a peptide that includes more than one cysteine residues, a natural or thiolated protein, thiolated hyaluronic acid, a thiolated collagen, and a combination of two or more thereof.

16. The composition of claim 15, wherein the thiol-containing molecule comprises a thiolated protein and the thiolated protein is thiolated gelatin.

17. A method for making a composition comprising a hydrogel, the method comprising:
forming a polymer of formula (1) via a radical process: 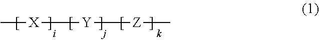
where
X comprises one or more repeating units of formula (2): 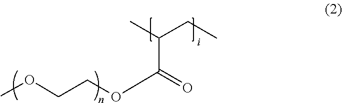
Y comprises one or more repeating units of formula (3): 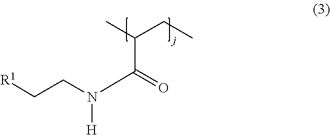
Z comprises one or more repeating units of formula (4): 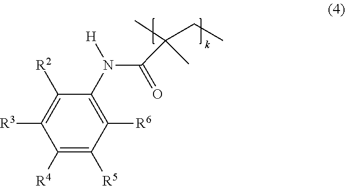
R¹ is H or OH;
R²-R⁶ each independently comprise H, C, or a heteroatom;
n, i, j, and k, are each independently an integer from 1 to 4000;
wherein
at least one repeating unit of Z comprises BO₂H₂; and
conjugating a norbornene of formula (5) to at least one repeating unit of Y to produce a norbornene-functionalized polymer of formula (1), where the substituent of formula (5) is substituted for R¹ via a position labeled *:

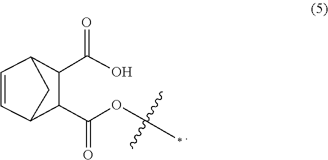

18. The method of claim 17, further comprising:
adding at least one pancreatic cancer cell to the composition.

19. The method of claim 18, wherein the pancreatic cancer cell is from a mammal.

20. The method of claim 19, wherein the mammal is a human.

21. A method for encapsulating at least one cell, the method comprising:
forming the composition of claim 1;
adding a thiol-containing molecule to the composition;
adding the cell to the composition; and
allowing the substituent of formula (5) to react with the thiol-containing molecule, thereby forming the hydrogel and encapsulating the cell.

22. The method of claim 21, wherein the cell is a cancer cell or wherein the cell is a pancreatic cancer cell or wherein the cell is a mammalian pancreatic cancer cell or wherein the cell is a human pancreatic cancer cell.

23. (canceled)

24. (canceled)

25. (canceled)

26. The method of claim 21, wherein allowing the substituent of formula (5) to react with the thiol-containing molecule comprises exposing the composition to electromagnetic energy.
